(19) United States
(12) Patent Application Publication
(10) Pub. No.: US 2014/0314670 A1
(43) Pub. Date: Oct. 23, 2014

(54) CLICKED SOMATOSTATIN CONJUGATED ANALOGS FOR BIOLOGICAL APPLICATIONS

(71) Applicants: CENTRE NATIONAL DE LA RECHERCHE SCIENTIFIQUE (CNRS), Paris cedex 16 (FR); UNIVERSITE DE BOURGOGNE DIJON, Dijon (FR); UNIVERSITE DE CERGY PONTOISE, Cergy Pontoise cedex (FR)

(72) Inventors: Débora D'Addona, Sesto Fiorentino (IT); Claire Bernhard, Dijon (FR); Anna Maria Rovero, Florence (IT); Franck Denat, Dijon (FR); Michael Chorev, Chestnut Hill, MA (US)

(21) Appl. No.: 14/365,875

(22) PCT Filed: Dec. 14, 2012
(86) PCT No.: PCT/IB2012/057310

§ 371 Date: Jun. 16, 2014

(30) Foreign Application Priority Data
Dec. 14, 2011 (EP) 11193530.0

(51) Int. Cl. A61K 51/08; A61K 47/48; A61K 51/04

(52) U.S. Cl. CPC A61K 51/083; A61K 51/088; A61K 51/0482; A61K 47/48076
USPC 424/1.69; 530/311; 514/11.1

(57) ABSTRACT

The invention relate to the new conjugated somatostatin analogs of formula (I), wherein A- is H or TAG-B—, wherein —B— is 0 or a spacer, wherein TAG is a chelating agent (e.g. DOTA), a fluorescent dye, (e.g. Bodipy-B derivative, a Rho-damine-B derivative, a Fluorescein-B derivative, a Cyanine-B derivative, a Porphyrin-B derivative), a bimodal agent, or a cytotoxic agent (e.g. a doxorubicin derivative) wherein R1 is CI-C4 alkynyl radical when R2 is CI-C4 azido radical and viceversa and methods for preparing same, pharmaceutical compositions comprising them and their use in imaging and the treatment of cancer.

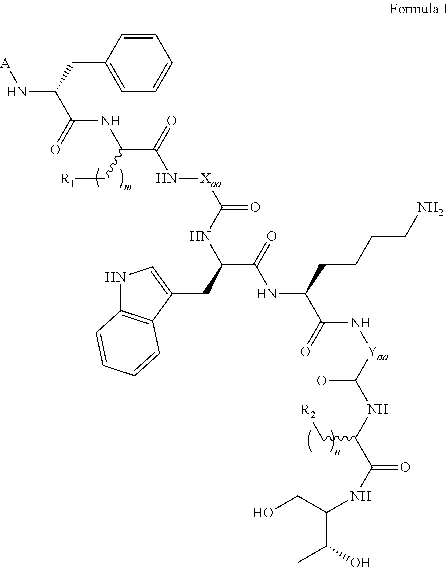

The present invention is directed to somatostatin conjugated analogs and their use for biological applications, particularly for diagnosis, prognosis, monitoring disease activity, and evaluation of efficacy of therapeutic treatments. It also relates to the therapeutical applications thereof, advantageously for the curative treatments of tumors expressing somatostatin receptors.

### INTRODUCTION

The goal of the present invention is to provide new conjugated somatostatin analogs to fulfill the unmet needs of clinicians interested in more selective and stable agents that can be used as: 1) diagnostic tools in different imaging techniques, to efficiently and specifically target tumors. 2) cytotoxic or radiolabeled molecules for specific and efficacious targeted therapy of cancer.

### BACKGROUND/PRIOR ART/DRAWBACKS

The peptide hormone Somatostatin SRIF (for Somatotropin Release—Inhibiting Factor) has formula (A)

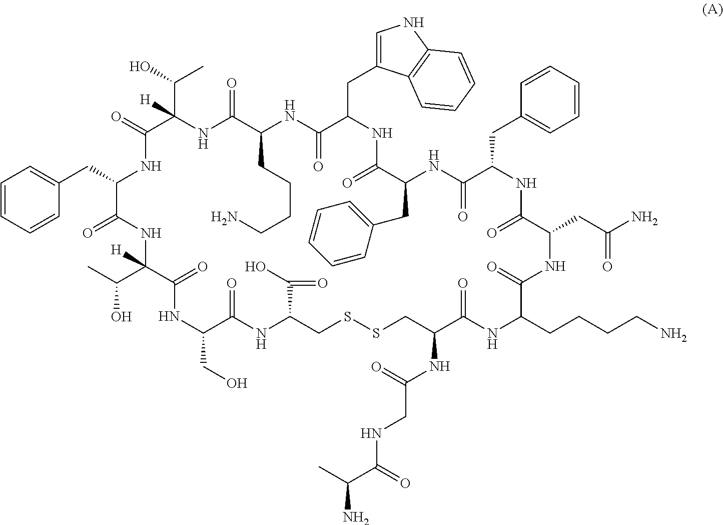 [SMILES: C[C@H](N)C(=O)NCC(=O)N[C@H]1CSSC[C@@H](C(=O)O)NC(=O)[C@H](CO)NC(=O)[C@H]([C@@H](C)O)NC(=O)[C@H](Cc2ccccc2)NC(=O)[C@H]([C@@H](C)O)NC(=O)[C@H](CCCCN)NC(=O)C(Cc2c[nH]c3ccccc23)NC(=O)[C@H](Cc2ccccc2)NC(=O)[C@H](Cc2ccccc2)NC(=O)[C@H](CC(N)=O)NC(=O)C(CCCCN)NC1=O]

It appears in two active forms, one of 14 and the other of 28 amino acids. SRIFs are side chain-to-side chain disulfide-bridged cyclic peptides. They are predominantly produced by neurones and secretary cells in the central and peripheral nervous system and in the gastrointestinal tract. SRIFs are unique in their broad inhibitory effects on both endocrine secretion of hormones such as growth hormone (GH), insulin, glucagon, gastrin, cholecystokinin, Vasoactive Intestinal Peptide (VIP), and secretin, and exocrine secretion of fluids such as gastric acid, intestinal fluid, and pancreatic enzymes. In addition, the distribution of SRIFs in central nervous system and in the spinal cord makes them an important player in neuronal transmission.

The biological effects of SRIFs, all inhibitory in nature, are mediated by a family of structurally related, G-protein-coupled transmembrane receptors. These are classified into SRIF₁ receptor subtypes: sstr₂, sstr₃, and sstr₅, and SRIF₂ receptor subtypes: sstr₁ and sstr₄.

The unique pharmacological effects of SRIF-14 are derived from its universal high-affinity binding to all somatostatin-receptor subtypes. To overcome the short-lived presence of SRIF-14 in circulation (plasma half-life of <3 min) many analogs consisting of cyclic peptides of 6 to 11 amino acids tethered by the disulfide bridge (Cys2-Cys7), have been developed in an attempt to stabilize the pharmacophoric 3-turn region. Despite the large number of products developed up to now, only octreotide and lanreotide are approved for clinical use and pasereotide is in late phase of clinical development. These drugs are long acting, with circulating half-lives of about 90 min. However, their clinical use is limited, because they lack considerable endocrine selectivity. This family of drugs is much more efficacious than SRIF-14 in inhibiting the release of GH, glucagon, and insulin. In humans, long-term treatment with SRIF analogs is sometimes associated with hyperglycemia due to their inhibitory effects on insulin secretion.

In the field of diagnosis and therapy of Somatostatin-positive tumors, the SRIF analogs Octreoscan® and OctreoTher® are the mostly used monomodal diagnostic radiotracers. These are disulfide-bridged octapeptide somatostatin analogs of octreotide and Tyr-3-octreotide (TOC), respectively. These cyclopeptides are modified at the N-terminus with radiochelators. In particular, Octreoscan® is modified with DTPA-¹¹¹In (DTPA, diethylene triamine pentaacetic acid) and Octreother® is modified with DOTA-⁹⁰Y (DOTA, 2,2′,″,2′″-(1,4,7,10-tetraazacyclododecane 1,4,7,10-tetrayptetraacetic acid).

Optimisation of these radiotracers for targeting tumoral cells requires increased bioavailability, better selectivity, and higher specificity. The following are some of the limitations presented by the current radiotracers that the invention is aiming to overcome:

The macrocyclic chelators should avoid: i) metal leaching into the body and consequently loss of selectivity/specific radioactivity; ii) high toxicity generally due to transchelation or transmetallation.

Moreover, the disulfide bridge present in SRIF analogs displays the following drawbacks: i) reduction of the disulfide by endogenous enzymes (i.e., by glutathione reductase and thioredoxin reductase); ii) cleavage by nucleophilic and basic agents; iii) interference with radiolabelling during the synthesis.

Intermolecular side chain-to-side chain cyclization is an established approach to achieve stabilization of specific conformations and a recognized strategy to improve resistance toward proteolytic degradation. Replacement of the disulfide bridge by end-to-end backbone cyclization as in the constraint analogs (i.e c[Phe-Pro-Phe-D-Trp-Lys-Thr]) were reported by Mattern and co workers. Despite a quite good affinity for sst sub-receptors they did not turn to be more potent or clinically useful somatostatin analogs.

Even if, the backbone cyclization substituting the disulfide bridge and proposed in a series of SRIF analogs, overcomes the side effect on hyperglycemia, a low affinity for some sst sub-receptors compared with octreotide makes this complex strategy not easily exploitable. Moreover, this cyclization linkage is still prone to endogenous cleavage (Afargan et al, Endocrinology 2001, 142, 477-486; Conformationally constrained backbone cyclized somatostatin analogs Hornik, Vered; Seri-Levy, Alan; Gellerman, Gary; Gilon, Chaim From PCT Int. Appl. (1998), WO 9804583 A1 19980205).

Octreotide analogs cyclized via the dicarba-linkage biolsosteric to the disulfide bridge (managing only one single ring dimension) was already proposed by two of the inventors of the present patent application (D'Addona et al. J. Med. Chem., 2008, 51, 512-520, Dicarba-analogues of octreotide WO2010004512A).

With these considerations in mind other side-chain to side-chain modifications shall be considered to introduce new bridging regions synthetically accessible (Le Chevalier Isaad A., Papini A. M., Chorev M., Rovero P. J. Pept. Sci. 2009; 15: 451-454) and less prone to oxidising and reducing attack.

The application of 1,2,3-triazoles has occurred only recently, following the discovery of regioselective Cu(I)-catalysed click chemistry in 2002. 1,2,3-triazoles show particular promise as amide bond isosteres, given their favourable pharmacophoric properties, excellent stability against isomerases and proteases and because of their accessible synthesis starting from a collection of synthetically available ω-alkynyl- and ω-azido-functionalised derivatives of chiral L and D amino acids (Formulae (B) and (C)) (A. Le Chevalier Isaad, F. Barbetti, P. Rovero, A. M. D'Ursi, M. Chelli, M. Chorev, A. M. Papini, Eur. J. Org. Chem., 2008, 31, 5308.)

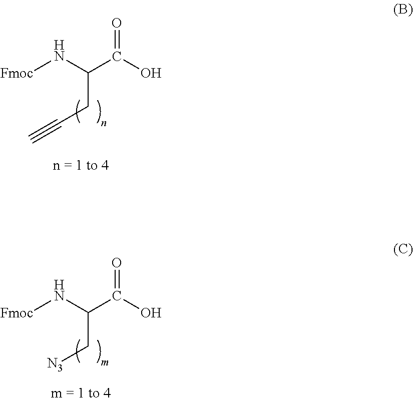

It is worth of note that, thanks to the different length of the alkynyl and azide amino acids side chains, the inventors of the present patent application were able to investigate not only the influence of the triazolyl moiety but also of the ring size on the bioactivity, selecting the right orientation, the number of methylene groups in amino-acid side chains to obtain more stable and specific analogues generating the optimal bioactive conformation.

Recently the incorporation of 1,2,3-triazoles was described: i) by Choi et al. as β-turn mimics into peptide nanotubes (W. J. Choi, Zhen-Dan Shi, a Karen M. Worthy, L. Bindu, Rajeshri G. Karki, M. C. Nicklaus, R. J. Fisher, T. R. Burke; Bioorganic & Medicinal Chemistry Letters-16-2006-5265-5269); ii) by Chorev and Papini for α-helical conformation stabilization (S. Cantel, A. Le Chevalier-Isaad, M. Scrima, J. J. Levy, R. D. DiMarchi, P. Rovero, J. A. Halperin, A. M. D'Ursi, A. M. Papini, M. Chorev; J. Org. Chem., 2008, 73, 5663-74); iii) by Jacobsen et al. to induce an 3₁₀-helical structure (Jacobsen Ø., † Maekawa H., Ge N.-H, Göorbitz H. C., Rongved P., Ottersen O. P., Amiry-Moghaddam M., Klaveness J.; J. Org. Chem. 2011, 76, 1228-1238) iv) and in the meanwhile we were filing this patent appeared online disulfide bond mimetic analogues (Meldal M.; Angew. Chem. Int. Ed. Online DOI: 10-1002).

We argue that in order to enhance the stability and availability of new SRIF analogs in vivo, the use of 1,2,3-triazole moieties leading to peptidomimetics reproducing native bioactive conformation (Scrima M.: Le Chevalier Isaad A.; Rovero P.; Papini A. M.; Chelli M.; Chorev, D'ursi A. M. Eur. J. Org. Chem., 2010, 3, 446-457) have to be considered.

In view of the closest prior art recited above, the present inventors identified that remains a need for new somatostatin analogs that overcome the difficulties and problems reviewed above.

### DETAILED DESCRIPTION OF THE INVENTION

The present invention enables to overcome the aforesaid problems by providing new conjugated somatostatin linear analogs of formula [I], J″

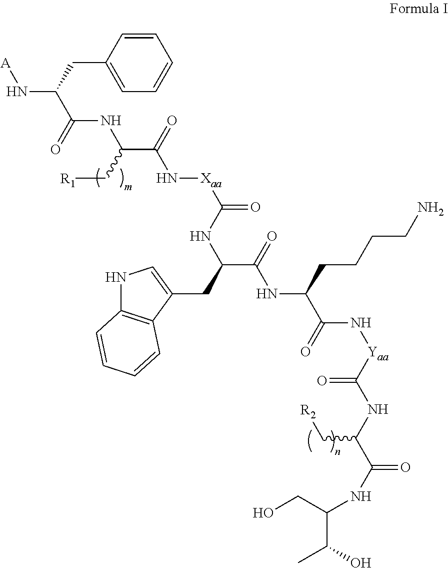

wherein A- is H or TAG-B—,
   wherein B— is 0 or a spacer of formula C-D-E,
      —C— and E are the same or different and are:
         a chain —(CH₂)ᵢ—W where i ranges from 0 to 20 and can be preferably 0 and 1 and W is 0, a chain containing 1 to 6 (C═O) or (C═S) group(s) and/or one or more heteroatoms (N, O, S, P), and/or one or 1 to 3 aromatic moiety(ies) containing zero, one or more heteroatoms (N, O, S, P), ortho and/or meta and/or para substituted or a chain containing one to 12 amino acid moieties choosing among the 22 natural α-amino acids, β or γ amino acids residues, preferably lysine or threonine. C is advantageously selected from the group comprising 0, CH₂—, CH₂CH₂—, CH₂Ar—, CH₂CH₂Ar—, CH₂CONHCH₂CH₂—, CH₂CONHCH₂CH₂NHCH₂—, CH₂NHCOCH₂Ar—, CH₂NHCOCHArNHCO CH₂—, CH₂NHCH₂ArOCH₂—. E is advantageously a group containing one amino acid moiety chosen among the 22 natural α-amino acids, β or γ amino acids residue or a group containing an unnatural amino acid modified on a amino or carboxyl group and/or in side chain position.
         a (PE_{G})ⱼ-W chain where j=2, 4, 100, 300, 1000 and W is as described above
      D being a group resulting from the coupling of two functional groups F and G present on the TAG and on the peptide respectively and defined as follows:
         D=thiourea generated from F=—NH₂ and G=—N═C═S (and vice versa), D=amide generated from F=NH₂ and G=COX (X=activated ester, Cl, anhydride, . . . ) (and viceversa), D=thioether generated from F=maleimide- and G=—SH (and vice versa); D=1,4-disubstituted [1,2,3]-triazole generated from F=—N═N⁺═N⁻ and G=-alkyne (and viceversa), D=imine generated from F=aldehyde and G=NH₂ (and vice versa). D=coupling function generated from F=norbornene or trans-cyclooctene and G=1,2,4,5 tetrazine (and viceversa).
   wherein TAG is
      a chelating agent CA
         wherein CA-B is one of the formula 1 to 8.
         R and R′ are groups capable of chelating a metal of interest, for either imaging purposes (Gd (MRI), ¹¹¹In or ⁶⁷Ga (SPECT), ⁶⁴Cu or ⁶⁸Ga (PET), . . . ) or therapy (⁹⁰Y, ¹⁷⁷Lu, ⁶⁷Cu, . . . ), preferentially COO⁻, COOH, P(O)(OH)Me or CONH₂.

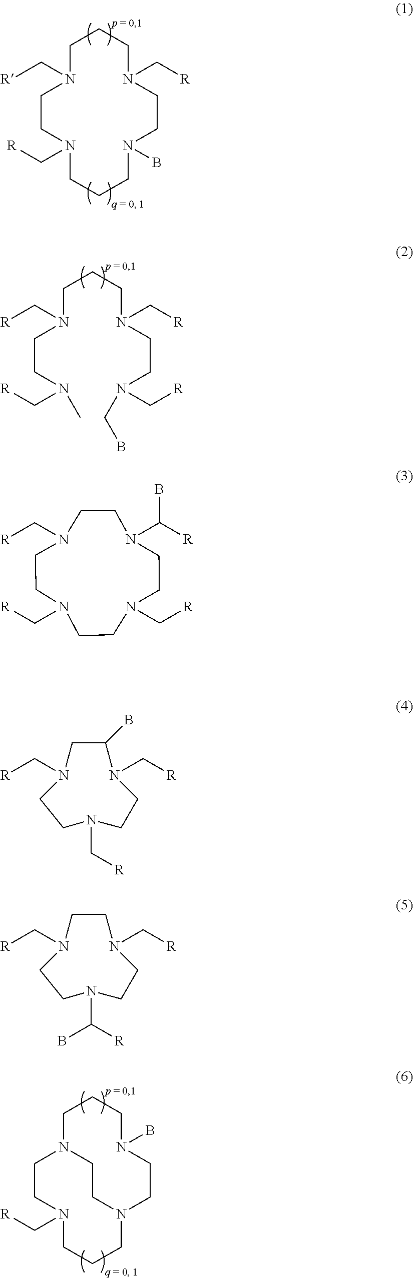

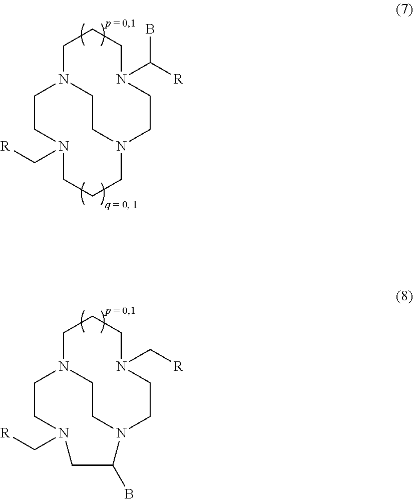

a fluorescent dye FD
   wherein FD-B is an organic fluorophore-B such as a Bodipy-B derivative, a Rhodamine-B derivative, a Fluorescein-B derivative, a Cyanine-B derivative, a Porphyrin-B derivative or a fluorescent complex of formulae (9) to (12) wherein 3 is a chromophore group and Ln is a Lanthanide chosen from Lanthanides giving high fluorescent Lanthanide complexes.

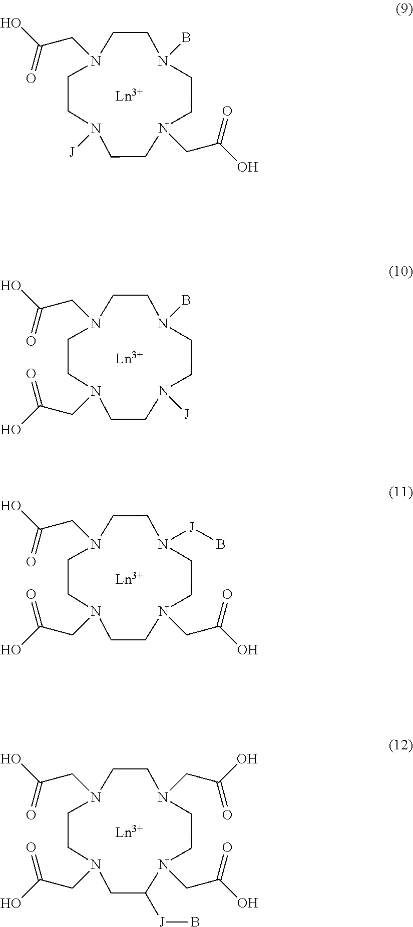

a bimodal agent BA comprising both a chelating agent CA and a fluorophore FD coupled together particularly using an amino acid linker, for example BA-B being one of the formula 13 or 14.

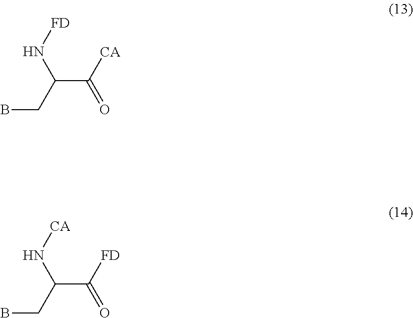

FD and B are the same as described previously.
a cytotoxic molecule CT
   wherein CT-B is a potent cytotoxic derivative of 2-pyrrolino-DOX (formula 15) or doxorubicin (DOX) of formula (16) capable to inhibit the growth of various tumors.

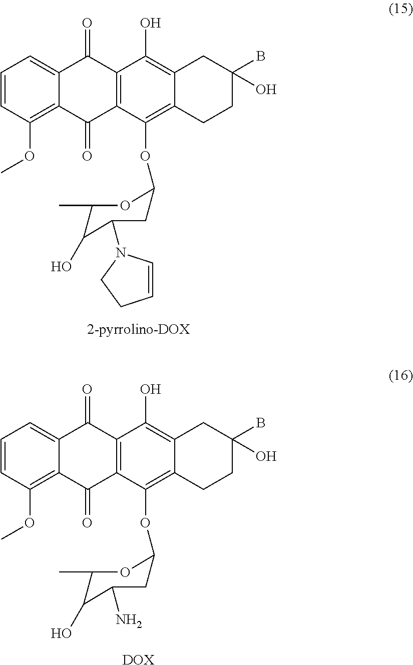

wherein Xaa is all at least one of the 22 L or D amino acids or phenylalanine either unsubstituted or ortho- or para-substituted with the OR″ group where, R″ is linear or branched C1-C6 alkyl substituted; otherwise Xaa is 1-NaI (NaI naphthylalanine) or 2-NaI as such substituted on the 1 or 2 ring by OR″ group where R″ is H or linear or branched C1-C6 alkyl substituted or a C₆H₅—CH₂—.
wherein Yaa is all 22 L or D amino acids or phenylalanine either unsubstituted or ortho- or para-substituted with the OR″ group where, R″ is linear or branched C1-C6 alkyl substituted or a threonine substituted with OR′″ where R′″ is H or a linear branched alkyl residue or a C₆H₅—CH₂— residue either unsubstituted or ortho- or para-substituted with the OR″ group, R″ being as above defined or a substituted C₆H₅—CH₂— or 1- or 2-naphthylmethyl-; otherwise Yaa 1-NaI or 2-NaI as such unsubstituted or substituted on the 1 or 2 ring by —OCH₃ or C₆H₅—CH₂—O—,
wherein R1 is Cᵣ C₄ alkynyl radical when R2 is Cᵣ C₄ azido radical and viceversa. m and n are the same or different and are 1 to 5.
   In a group of analogues of the present invention, the compounds have formula (II), where R1 and R2 form an intramolecular [1,2,3]triazolyl bridge T.

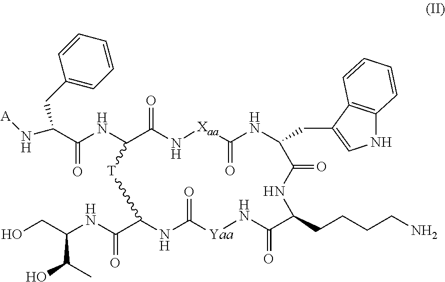

Wherein A, Xaa, Yaa are the same as described above, The orientation of the 1,4 disubstituted [1,2,3]triazolyl moiety depends on the position of ω-azido or ω-alkynyl aminoacids in the peptide chain, respectively i+5 and viceversa (formulae 1 and 2).

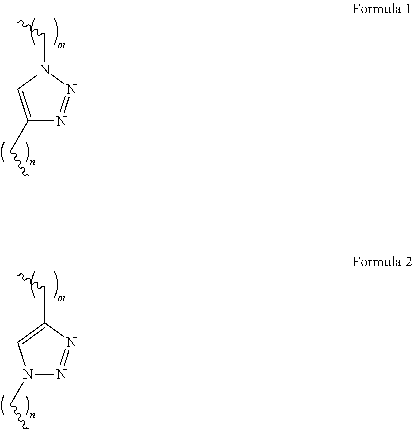

Analogs of formulae (I) and (II) wherein TAG-B is CA B chelated with ¹¹¹In, ^{67/68}Ga or ⁶⁴Cu or FD-B or BA-B are more particularly useful for imaging.

Analogs of formulae (I) and (II) wherein TAG-B is CA-B and chelated with for example ⁹⁰Y, ¹⁷⁷Lu or ⁶⁷Cu are useful in therapy.

Analogs of formulae (I) and (II) wherein A is H can be useful in therapy.

Likewise, analogs of formulae (I) and (H) wherein TAG is a cytotoxic molecule CT are of high value to inhibit the growth of various tumors.

The compounds of formulae (I) or (II) are advantageously prepared starting from known or easily prepared products.

The invention thus relates to a method for preparing the analog derivatives of formula (I):
Solid Phase Peptide Synthesis (SPPS), comprising anchoring a peptide sequence to a derivatized chlorotrityl resin
Recovering the crude peptide by treating the resin for example with trifluoroacetic acid
Separating by precipitation for example ethyl ether
Carrying out successive lyophilization
Purifying the peptide using chromatographic techniques for example RP-HPLC

More particularly, the specific sequence by anchored to a derivatized chlorotrityl resin H-L-Thr(t-Bu)-ol-2-chlorotrityl resin. Upon completion of the synthesis the crude peptide were obtained by treating the resin with trifluoroacetic acid and separated by precipitation with ethyl ether and successive lyophilization. The peptide is finally purified using chromatographic technique, such as for example RP-HPLC.

The invention also relates to a method for preparing the derivative of formula (II) comprising
a) Synthesizing a linear heptapeptide following the Fmoc/tBu SPPS strategy with appropriate side chain protected aminoacids.
b) Cyclising the peptides linked to the resin by means of Cu(I)-catalyzed azide-alkyne 1,3-dipolar Huisgen's cycloaddition to form regioselective 1,4-disubstituted-[1,2,3]triazolyl bridge
and either
c) adding the N-terminus aminoacid and the group A defined as described above in solid phase or alternatively following the solution strategy described in step c′ to e′.
d) cleaving the conjugated 1,4-disubstituted-[1,2,3]triazolyl bridge containing peptides from the resin with an appropriate acid.
e) purifying by semipreparative RP-HPLC the crude 1,4-disubstituted-[1,2,3]triazolyl containing cyclic conjugated peptides.
or
c′) adding the N-terminus amino acids and/or the group E-G, defined as described above; cleaving the peptide from the resin with an appropriate acid.
d′) coupling the group TAG in solution to the 1,4-disubstituted-[1,2,3]triazolyl bridge containing peptides.
e′) purifying by semipreparative RP-HPLC the crude 1,4-disubstituted-[1,2,3]triazolyl bridge containing peptides conjugated.

When A represents TAG-B, the method further comprises conjugating the resulting peptide to a tag derivative of formula (III)

TAG-C—F (III)

Wherein C and F are the same as described previously

The biological studies of the conjugated somatostatin linear analogs of formula [I] and 1,4-disubstituted [1,2,3]-triazolyl bridged somatostatin cyclopeptide analogs of formula [II] have shown these derivatives are of great interest in imaging and therapy of cancer.

They are able to deliver effectively, specifically and with minimal loss tags and therapeutic cytotoxic molecules to affected sites and organs to achieve optimal imaging or therapy of cancer.

Several features of the new cyclopeptide analogs contribute to confer to them a high selectivity and affinity for the different subtypes SST receptors, particularly, their bridging region with a bioisosteric heterocyclic moiety, the optimization of the length of the bridge and the location and orientation of the heterocyclic moiety in the bridge.

According to a first aspect, the invention thus relates to the use of the above defined compounds as radiotracers for imaging tumoral cells.

It particularly relates to a method of radio-isotoping imaging comprising the use of at least one compound according to formula (I) or (II) wherein A represents TAG-B.

In said method, the compound is administered by injection.

This method is particularly useful for SPECT/PET imaging and/or optical imaging. Mixing SPECT or PET with optical imaging enable detection by two imaging techniques and thus provide useful complementary diagnostic information.

According to a second aspect, due to their high binding affinity to sst 1-5 receptors, the compounds of the invention are therapeutic agents of interest as inhibitors for treating cancers, for example lymphoma, pancreatic, lung, prostate or breast cancer, or adenoma.

The invention thus also relates to pharmaceutical compositions comprising a therapeutically efficient amount of at least one compound of formulae (I) and (II) in combination with a pharmaceutically acceptable carrier.

The dosage in the pharmaceutical preparations will be easily determined by the one skilled in the art in view of the pathology to be treated. The doses per dosage unit will be chosen depending on the condition and age of the patient.

Examples disclosing the preparation of some conjugated peptides and chelating agents, according to the invention, are provided as follows to illustrate the purposes of the invention.

1) Synthesis of TAG where TAG=CA, Containing Three Different Groups F

### Example 1

Synthesis of compound 1 where F is an acid function (1 step starting from the commercially available tri-tert-butyl 2,2′,2″-(10-(2-((2-aminoethyl)amino)-2-oxoethyl)-1,4,7,10-tetraazacyclododecane-1,4,7-triyl)triacetate).

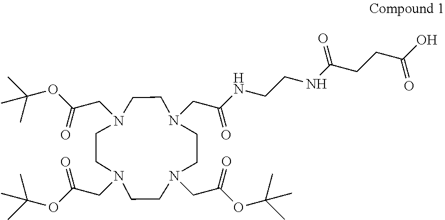 [SMILES: CC(C)(C)OC(=O)CN1CCN(CC(=O)NCCNC(=O)CCC(=O)O)CCN(CC(=O)OC(C)(C)C)CCN(CC(=O)OC(C)(C)C)CC1]

100 mg of succinic anhydride (1 mmol) were added to a solution of tri-tert-butyl 2,2′,2″-(10-(2-((2-aminoethyl)amino)-2-oxoethyl)-1,4,7,10-tetraazacyclododecane-1,4,7-triyl)triacetate (620 mg, 1 mmol) in 10 mL of 1-4 Dioxane. The mixture was stirred at room temperature for 4 h. After evaporation of the solvent, the solid was taken in diethyl ether and filtered. The solvent was evaporated to give the compound 1 as a white foam (m=680 mg, yield=94%). ¹H NMR (300 MHz, CDCl₃, 300 K) δ(ppm): 1.41 (s, 27H, OC(CH₃)₃), 2.37-2.46 (m, 2H), 2.53-3.42 (m, 26H), 3.53-3.55 (m, 2H), 3.86-3.95 (m, 2H), 7.45 (bs, 1H, NH), 8.88 (bs, 1H, NH), 9.52 (bs, 1H, COOH). ¹³C{¹H} NMR (75 MHz, CDCl₃, 300 K) δ (ppm): 28.3 (*9) (CH₃), 32.5, 32.7, 38.8, 39.4, 48.9 (*2), 50.8 (*2), 53.5 (*2), 53.7 (*2), 55.6, 56.1, 56.9 (*2) (CH₂), 81.8, 81.9 (*2) (C), 170.2 (*2), 170.4, 174.2, 177.8, 177.9 (C═O). MALDI-TOF: m/z=715.27 [M+H]⁺, 737.944 [M+Na]⁺. Elemental analysis: C₃₄H₆₂N₆O₁₀.2H₂O C₄H₈O₂. Calculated: C (54.40%), H (8.89%), N (10.42%). Obtained: C (54.32%), H (8.73%), N (10.02%).

### Example 2

Synthesis of compound 2e where F is an isothiocyanate function (6 steps starting from the macrocycle 5-aminomethyl13aneN4 prepared according to the literature (Rousselin, Y.; Sok, N.; Boschetti, F.; Guilard, R.; Denat, F. Eur. J. Org. Chem. 2010, 1688).

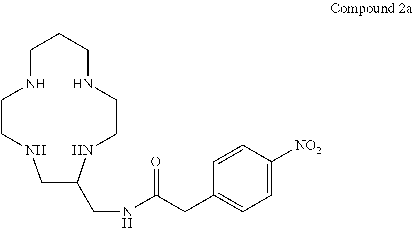 [SMILES: O=C(Cc1ccc([N+](=O)[O-])cc1)NCC1CNCCNCCCNCCN1]

4.27 g of N-succinimidyl-4-nitrophenylacetate (1.53 mmol) were added to a solution of 5-aminomethyl13aneN4 (3.3 g, 1.53 mmol) in dichloromethane (20 mL). The reaction mixture was stirred at room temperature for 2 h. Solvent was evaporated, the weak pink foam was dissolved in ethanol (10 mL) and a solution of HCl 35% (20 mL) was added. The precipitate was filtered and washed with ethanol (2*30 mL). The compound 2a (+4HCl) was obtained as a white solid, which could be recrystallized in a mixture of water/ethanol. The resulting solid was dissolved rapidly in 15M NaOH solution until pH=14. After extraction with chloroform (2*100 mL), the organic phase was dried over MgSO₄ and the solvent was evaporated to give a pink oil. This oil was taken in dichloromethane (10 mL), and upon addition of pentane (150 mL) a precipitate was formed slowly. The solution was left standing overnight to complete reprecipitation. The product was filtered, washed with pentane, and dried in vacuo to give 2a as a pink solid (m=4.40 g, yield=76%). ¹H NMR (300 MHz, CDCl₃, 300 K) δ (ppm): 1.48-1.76 (m, 2H, CH₂-β), 2.33-2.90 (m, 19H), 3.12-3.31 (m, 2H), 3.59 (s, 2H, CH₂Ar), 6.79 (bs, 1H, NIA 7.74 (d, 2H, ³J=8.6 Hz), 8.12 (d, 2H, ³J=8.6 Hz). ¹³C{¹H} NMR (75 MHz, CDCl₃, 300 K) δ (ppm): 28.8 (CH₂-β), 43.4, 46.0, 47.6, 48.7, 48.9, 49.9, 50.5 55.6 (CH₂), 55.8 (CH), 65.9 (CH₂Ar), 123.9 (*2), 130.3 (*2) (CHₐᵣ)_{,} 143.1, 147.8 (Cₐᵣ), 169.2 (C═O). MALDI-TOF: m/z=379.24 [M+H]⁺, 401.23 [M+Na]⁺. Elemental analysis: C₁₈H₃₀N₆O₃, 3HCl, MeOH. Calculated: C (43.89%), H (7.17%), N (16.16%). Obtained: C (44.21%), H (7.01%), N (16.28%).

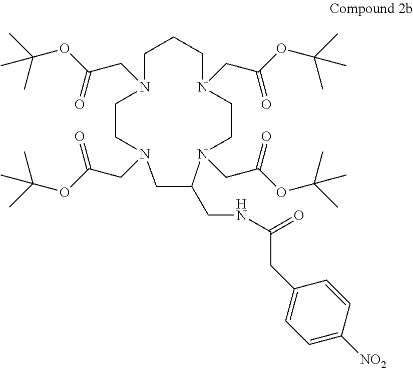 [SMILES: CC(C)(C)OC(=O)CN1CCCN(CC(=O)OC(C)(C)C)CCN(CC(=O)OC(C)(C)C)C(CNC(=O)Cc2ccc([N+](=O)[O-])cc2)CN(CC(=O)OC(C)(C)C)CC1]

A solution of tertbutylbromoacetate (3.1 g, 16 mmol) was added to a solution of 2a (1.5 g, 3.9 mmol) and K₂CO₃ (3.7 g, 27 mmol) in acetonitrile (50 mL). The resulting mixture was heated at 45° C. overnight. After cooling, the solution was filtered on celite and the solvent was evaporated and the resulting oil was taken in ether. The mixture was filtered, the solvent evaporated, and the residue was purified to chromatography on aluminium oxide (eluent: CH₂Cl₂/MeOH 99:1) to give compound 2b as a yellow oil (1.6 g, yield=48%). ¹H NMR (300 MHz, CDCl₃, 300 K) δ(ppm): 1.37-1.45 (m, 37H), 2.37-2.50 (m, 2H), 2.52-3.01 (m, 14H), 3.11-3.26 (m, 8H), 3.34-3.47 (m, 2H), 3.63 (s, 2H, CH₂Ar), 7.50 (d, 2H, ³J=8.6 Hz), 7.88 (bs, 1H, NH), 8.12 (d, 2H, ³J=8.6 Hz). ¹³C{¹H} NMR (75 MHz, CDCl₃, 300 K) δ(ppm): 25.2 (CH₂-β), 28.2 (*3), 28.4 (*9) (CH₃), 40.1, 43.5, 49.4, 49.9, 50.8, 52.3, 52.6, 53.1, 53.6, 55.1 (CH₂), 55.9 (CH), 56.8, 57.2, 57.9 (CH₂), 80.9 (*2), 81.1, 81.5 (C), 123.8 (*2), 130.4 (*2) (CHₐᵣ), 143.1, 147.0 (Cₐᵣ), 169.4, 171.0, 171.2, 171.5, 173.1 (C═O). MALDI-TOF: m/z=857.31 [M+Na]⁺.

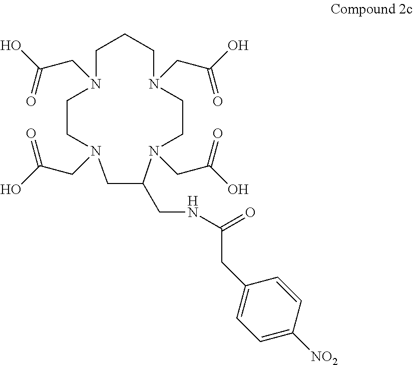 [SMILES: O=C(O)CN1CCCN(CC(=O)O)CCN(CC(=O)O)C(CNC(=O)Cc2ccc([N+](=O)[O-])cc2)CN(CC(=O)O)CC1]

800 mg of compound 2b (95.8 mmol) were dissolved in 8 mL of HCl 35%, The mixture was stirred for 30 min at room temperature. The mixture was evaporated to dryness to give a brown solid, which was taken in 10 mL of acetone and stirred at room temperature overnight. The precipitate was filtered, washed with ethanol, acetone, ether and finally dried in vacuum. The compound 2c (+3HCl) was obtained as a white solid (m=600 mg, yield=95%). ¹H NMR (300 MHz, D₂O, 300 K) δ(ppm): 2.20-2.22 (m, 2H, CH₂-β), 2.85-3.96 (m, 25H), 4.02-424 (m, 2H), 7.47 (d, 2H, ³J=8.65 Hz), 8.25 (d, 2H, ³J=8.65 Hz). ESI-MS: m/z=609.25 [M−H]⁻

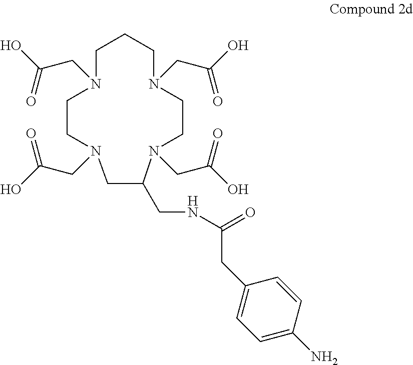 [SMILES: Nc1ccc(CC(=O)NCC2CN(CC(=O)O)CCN(CC(=O)O)CCCN(CC(=O)O)CCN2CC(=O)O)cc1]

Compound 2c (100 mg, 0.14 mmol) was placed in 8 mL of water and 6 mg of 10% Pd/C (5.58 μmol, 0.04 equivalent) was added under H₂. After consumption of the hydrogen, the suspension was eliminated by filtration on Clarcel® and the solvent was evaporated to give 2d (+4HCl) as a yellow solid (m=90 mg, yield=90%). ¹H NMR (300 MHz, D₂O, 300 K) δ(ppm): 2.06-2.10 (m, 2H, CH₂-β), 2.78-4.02 (m, 27H), 7.17-7.37 (m, 4H). ESI-MS: m/z=579.25 [M−H]⁻

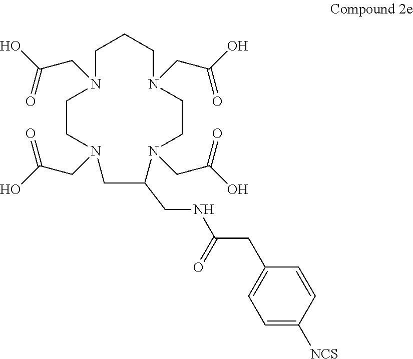

A solution of thiophosgene (30.4 μL, 0.41 mmol, 6 equivalents) in dichloromethane (2 mL) was added to a solution of 2d (50 mg, 68.8 μmol) in water (8 mL). After stirring vigorously for 2 h at room temperature, the resulting solution was washed with dichloromethane, the aqueous extracts were separated, and the solvent was evaporated. Compound 2e (+3HCl) was isolated as a yellow solid (m=48 mg, yield=98%). ¹H NMR (300 MHz, D₂O, 300 K) δ(ppm): 2.06-2.26 (m, 2H, CH₂-β), 2.76-4.26 (m, 27H), 7.01-7.52 (m, 4H). m/z=323.11 [(M+Na)/2]²⁺, 623.24 [M+H]⁺, 645.22 [M+Na]⁺

### Example 3

Synthesis of compound 3b where F is an alkyne function (2 steps starting from tri-tert-butyl 2,2′,2″-(10-(2-((2-aminoethyl)amino)-2-oxoethyl)-1,4,7,10-tetraazacyclododecane-1,4,7-triyl)triacetate).

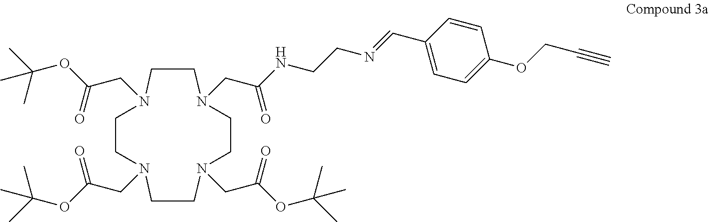 [SMILES: C#CCOc1ccc(/C=N/CCNC(=O)CN2CCN(CC(=O)OC(C)(C)C)CCN(CC(=O)OC(C)(C)C)CCN(CC(=O)OC(C)(C)C)CC2)cc1]

134 mg of 4-(prop-2-yn-1-yloxy)benzaldehyde (0.84 mmol) were added to a solution of tri-tert-butyl 2,2′,2″-(10-(2-((2-aminoethyl)amino)-2-oxoethyl)-1,4,7,10-tetraaza cyclododecane-1,4,7-triyl)triacetate) (517 mg, 0.84 mmol) in ethanol (10 mL) and the mixture was stirred at room temperature for 12 h. The solvent was evaporated to dryness, and the residual oil was taken in pentane. After stirring during 12 h, the insoluble impurities were removed by filtration. After evaporation of the solvent, the compound 3a was obtained as a white foam (m=530 mg, yield=84%). ¹H NMR (300 MHz, CDCl₃, 300 K) δ (ppm): 1.39 (s, 18H, OC(CH₃)₃), 1.41 (s, 9H, OC(CH₃)₃), 2.32-2.44 (m, 4H), 2.46 (t, 1H, ⁴J=2.4 Hz, CH₂—C≡CH), 2.50-2.79 (m, 12H), 2.90 (s, 2H), 3.06 (s, 4H), 3.20 (s, 2H), 3.40 (td, 2H, ³J=6.0 Hz, ³J=6.4 Hz, CH₂—CH₂—CH═N—), 3.61 (t, 2H, ³J=6.4 Hz, CH₂—CH₂—CH═N—), 4.58 (d, 2H, ⁴J=2.4 Hz, CH₂—C≡CH), 6.85 (d, 2H, ³J=8.5 Hz), 7.54 (d, 2H, ³J=8.5 Hz), 8.09 (s, 1H, N═CH—), 8.63 (t, 1H, ³J==6.0 Hz, NH). ¹³C{¹H} NMR (150 MHz, CDCl₃, 300 K) δ (ppm): 27.2 (*9) (CH₃), 39.5, 50.9 (*2), 51.3 (*2), 52.6 (*2), 53.9 (*2), 54.8, 55.2 (*2), 55.3, 57.2, 59.4 (CH₂), 75.0 (CH), 77.1, 79.6, 79.8 (*2) (C), 113.8 (*2), 128.6 (*2) (CHₐᵣ), 128.9, 158.5 (Cₐᵣ), 160.4 (N═CH), 169.5, 169.6 (*2), 171.3 (C═O). MALDI-TOF: m/z=779.43 [M+Na]⁺. HRMS-ESI: m/z=calculated for C₄₀H₆₄N₆O₈+Na: 779.4678, obtained 779.467.

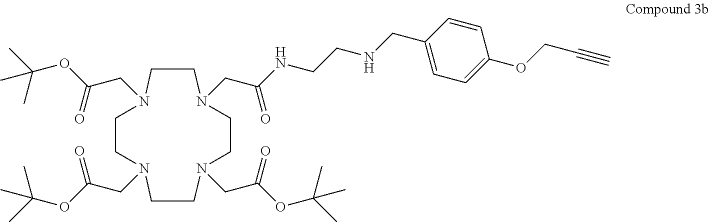 [SMILES: C#CCOc1ccc(CNCCNC(=O)CN2CCN(CC(=O)OC(C)(C)C)CCN(CC(=O)OC(C)(C)C)CCN(CC(=O)OC(C)(C)C)CC2)cc1]

15 mg of NaBH₄ (0.38 mmol) were added to solution of 3a (0.9 g, 0.19 mmol) in ethanol (20 mL) to 0° C. The mixture was stirred overnight at room temperature. The solvent was evaporated, the resulting solid was dissolved in dichloromethane (20 mL). After filtration of the insoluble impurities, the solution was washed with a 1M NaOH solution (5 mL), dried over MgSO₄ and the solvent was evaporated to give 3b as a very hygroscopic white foam (m=14 mg, yield=80%). ¹H NMR (300 MHz, CDCl₃, 300K) δ(ppm): 1.29-1.42 (m, 27H, OC(CH₃)₃), 1.97-3.15 (m, 25H), 2.46 (t, 1H, ⁴J=2.4 Hz, CH₂—C≡CH), 3.27-3.34 (m, 4H), 3.67 (bs, 2H), 4.60 (d, 2H, ⁴J=2.4 Hz, CH₂—C≡CH), 6.83 (d, 2H, ³J=8.5 Hz), 7.22 (d, 2H, ³J=8.5 Hz), 8.95 (bs, 1H, NH). ¹³C{¹H} NMR (75 MHz, CDCl₃, 300K) δ(ppm): 27.9 (*3), 28.2 (*6) (CH₃), 39.0, 48.3, 50.0, 52.1, 52.6, 55.7, 55.8, 56.1, (16*CH₂), 75.3 (CH), 78.2, 79.6, 81.7, 81.8 (C), 113.8 (*2), 129.5 (*2) (CHₐᵣ), 133.9, 158.4 (Cₐᵣ), 171.8, 171.9 (*2), 172.4 (C═O). ESI-MS: m/z=781.49 [M+Na]⁺. HRMS-ESI: m/z=calculated for C₄₀H₆₆N₆O₈+Na: 781.4834, obtained 718.4822.

### 2) Synthesis of TAG Where TAG=BA

### Example 4

Synthesis of compound 4e where F is an isothiocyanate function (5 steps starting from compound 4a).

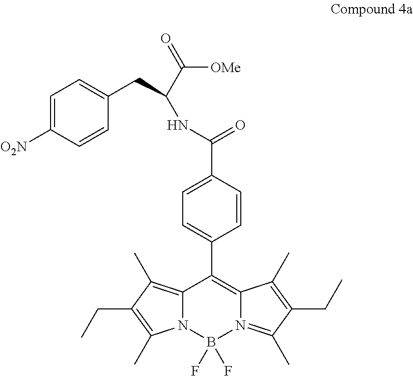

N-hydroxybenzotriazole (180 mg, 1.3 mmol), diisopropylethylamine (DIPEA) (340 mg, 2.6 mmol), 1-(3-dimethylaminopropyl)-3-ethylcarbodiimide hydrochloride (EDCI) (250 mg, 1.3 mmol) and 4-nitrophenylalanine methylester hydrochloride (340 mg, 1.3 mmol) were successively added to a solution of 4-carboxyphenyl-1,3,5,7-tetramethyl-2,6-diethyl-4-bora-3a,4a-diaza-s-indacene (550 mg, 1.3 mmol) in dry DMF (30 mL), and the solution was stirred at room temperature. After total consumption of the starting material (8 hours) followed by TLC (AcOEt/hexane 6:4, Rf=0.6), the solvent was evaporated. The solid obtained was washed with water (2*30 mL) and extracted with dichloromethane (100 mL). The organic phase was dried over MgSO₄ and the solvent was evaporated to give a red oil. The crude product was purified by column chromatography on silica gel (AcOEt/hexane 1:1). Recrystallization in CH₂Cl₂/hexane gave pure 10 as red-green crystals (630 mg, 76%). ¹H NMR (300 MHz, CDCl₃, 300K) δ(ppm): 0.93 (t, 6H, ³J=7.5 Hz), 1.23 (s, 6H), 2.28 (q, 4H, ³J=7.5 Hz), 2.51 (s, 6H), 3.34 (dd, 1H, ³J=13.9 Hz, 6.2 Hz), 3.45 (dd, 1H, J=13.9 Hz, 5.1 Hz), 3.79 (s, 3H), 5.14 (ddd, 1H, ³J=7.0 Hz, ³J=6.2 Hz, ³J=5.1 Hz), 6.75 (d, 1H, ³J=7.0 Hz), 7.33 (d, 2H, ³J=8.7 Hz), 7.40 (d, 2H, ³J=8.2 Hz), 7.88 (d, 2H, ³J=8.3 Hz); 8.15 (d, 2H, ³J=8.7 Hz); ¹³C{¹H} NMR (75 MHz, CDCl₃, 300K) δ(ppm): 11.9, 12.6, 14.6, 17.1, 37.9, 52.8, 53.5, 123.8, 127.7, 129.1, 130.2, 130.3, 133.2, 133.7, 138.1, 138.5, 140.0, 143.7, 147.3, 154.4, 166.2, 171.6; ¹¹B NMR (128 MHz, CDCl₃, 300K): 0.78 ppm (t, J_{B,F}=33.4 Hz); UV-Vis (CH₃CN), λ (nm) (ε, M⁻¹ cm⁻¹): 523 (63000), 492 (21100), 378 (6800); HRMS-ESI m/z; calcd for C₃₄H₃₇BF₂N₄O₅+H, 631.2903; found: 531.2897. Anal. Calcd for C₃₄H₃₇BF₂N₄O₅+0.3 CH₂Cl₂: C, 63.56; H, 5.82; N, 8.65; found: C, 63.44; H, 6.25; N, 8.63.

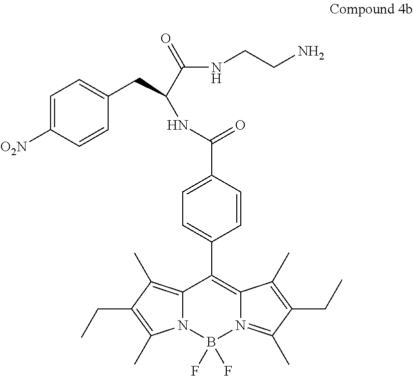

A solution of compound 4a (1.7 g, 2.7 mmol) and ethylenediamine (11.3 g, 0.18 mol) in 90 mL of methanol was stirred at 55° C. for 48 h. The solvent was evaporated, water was added (100 mL) and the product was extracted with dichloromethane (2*300 mL). The organic phase was dried over MgSO₄ and the solvent was evaporated. The crude product was washed with hexane (200 mL), and the solid obtained was purified by column chromatography on silica gel (CH₂Cl₂/MeOH/NH₄OH 80:18:2). Recrystallization in CH₂Cl₂/hexane gave pure 4b as red crystals (1.4 g, 79%). ¹H NMR (300 MHz, CDCl₃, 300K) δ(ppm): 0.95 (t, 6H, ³J=7.5 Hz), 1.22 (s, 6H), 2.27 (q, 4H, ³J=7.5 Hz), 2.51 (s, 6H), 2.64-2.81 (m, 2H), 3.18-3.36 (m, 2H), 4.87 (ddd, 1H, ³J=7.0 Hz, ³J=6.2 Hz, ³J=5.1 Hz), 6.36 (t, 1H, ³J=5.5 Hz, NH), 7.09 (d, 1H, ³J=7.0 Hz, NH), 7.38 (d, 2H, ³J=8.2 Hz), 7.45 (d, 2H, ³J=8.5 Hz); 7.88 (d, 2H, ³J=8.2 Hz), 8.16 (d, 2H, ³J=8.5 Hz); ¹¹B NMR (128 MHz, CDCl₃, 300K): 0.77 ppm (t, J_{B,F}=33.3 Hz); UV-Vis (CH₂Cl₂), λ (nm) (ε, M⁻¹ cm-¹): 528 (76500), 493 (23300), 379 (9650); ESI-MS: m/z=639.32 [M-F]⁺, 659.33 [M+H]⁺; Anal. Calcd for C₃₅H₄₁BF₂N₆O₄+0.7CH₂Cl₂: C, 59.72; H, 5.95; N, 11.70; found: C, 60.05; H, 5.79; N, 11.34.

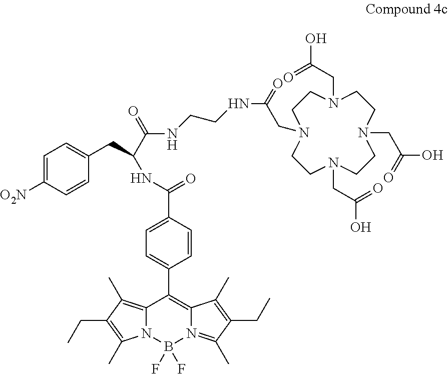

To a solution of compound 4b (230 mg, 0.34 mmol) and 170 μL of Et₃N (6 equivalents) in dry DMF (25 mL) was added a solution of DOTA-NHS ester (200 mg, 0.28 mmol) in dry DMF (5 mL). The mixture was stirred at room temperature for 12 h. Then the solvent was evaporated and the crude product was purified by column chromatography on silica gel (EtOH/NH₄OH 9:1). The solid obtained was washed with hexane (20 mL) and acetonitrile (20 mL). The compound 4c was isolated as a red solid (230 mg, 65%). ¹H NMR (600 MHz, MeOD, 330K) δ(ppm): 1.01 (t, 6H, ³J=7.5 Hz), 1.31 (s, 6H), 2.36 (q, 4H, 3J=7.5 Hz), 2.49 (s, 6H), 2.92-3.02 (m, 4H), 3.05-3.15 (m, 4H), 3.34-3.56 (m, 18H), 3.66-3.77 (m, 4H), 5.01-5.06 (m, 1H), 7.41 (d, 2H, ³J=8.2 Hz), 7.63 (d, 2H, ³J=8.5 Hz); 7.99 (d, 2H, ³J=8.2 Hz), 8.12 (d, 2H, ³J=8.5 Hz); ¹¹B NMR (128 MHz, MeOD, 300K): 0.72 ppm (t, J_{B,F}=33.2 Hz); UV-Vis (DMF), λ (nm) (ε, M⁻¹cm⁻¹): 523 (64400), 491 (21400), 379 (6800); ESI-MS: m/z=1067.49 [M+Na]⁺, 1089.47 [M+2Na−H]⁺, 1111.46 [M'3Na−2H]⁺, Anal. Calcd for C₅₁H₆₇BF₂N₁₀O₁₁+6.5H₂O, NH₄: C, 51.91; H, 7.17; N, 13.06; found: C, 51.75; H, 6.53; N, 12.38.

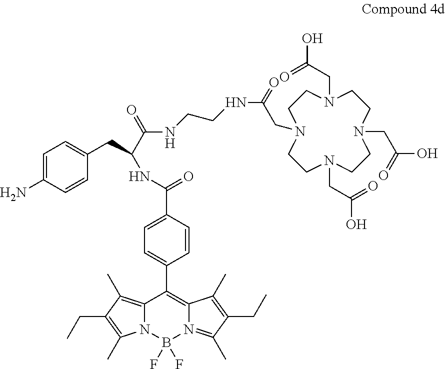

A suspension of compound 4c (50 mg, 47.8 μmol) and 10% Pd/C (5 mg, 19.2 μmol) in a mixture of water and ethanol (H₂O/EtOH, 90/10, 5 mL) was stirred under H₂. After consumption of hydrogen, the suspension was eliminated by filtration on Clarcel® and the solvent was evaporated. The solid obtained was washed with hexane (10 mL), to give compound 4d as a red solid (45 mg, 95%). ¹H NMR (300 MHz, MeOD, 300K) δ(ppm): 0.88 (t, 6H, ³J=7.5 Hz), 1.18 (s, 6H), 2.24 (q, 4H, ³J=7.5 Hz), 2.37 (s, 6H), 2.62-3.05 (m, 8H), 3.20-3.70 (m, 22H), 4.54-4.63 (m, 1H), 6.57 (d, 2H, ³J=8.2 Hz), 6.96 (d, 2H, ³J=8.5 Hz); 7.33 (d, 2H, ³J=8.2 Hz), 7.88 (d, 2H, ³J=8.5 Hz); ¹¹B NMR (128 MHz, MeOD, 300K): 0.71 ppm (t, J_{B,F}=33.2 Hz); UV-Vis (DMF), λ (nm) (ε, M⁻¹cm⁻¹): 523 (60200), 491 (19200), 379 (5600); ESI-MS: m/z=1051.43 [M+K−2H]⁻

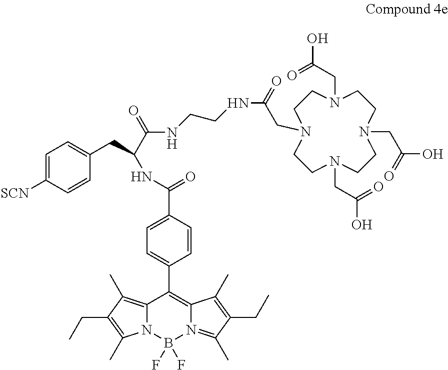

To a solution of 4d (15 mg, 14.7 μmol) in H₂O (5 mL) was added at room temperature a solution of thiophosgene (3.5 μL, 44.3 μmol) in chloroform (2 mL). The solution was stirred vigorously during 2 h. The solvent was evaporated and the residue was lyophilized. The crude product was washed with CH₂Cl₂ (2 mL) and hexane (5 mL) to give 4e as a red solid (13.5 mg, 90%); UV-Vis (DMSO), λ (nm) (ε, M⁻¹ cm⁻¹): 523 (43000), 492 (14400), 378 (4600); ESI-MS: m/z=1077.45 [M+Na−2H]⁻; 1093.41 [M+K−2H]⁻; 1099.42 [M+2Na−3H]⁻

3) Synthesis of linear conjugated octapeptide of Formula (I) where A is DOTA;

m=n=2; Xaa=—CH—CH₂—C₆H₅—OH; Yaa=—CH—CH(OH)—CH₃; R1=—N═N⁺═N⁻; R2=alkynyl [DOTA-D-Phe-Abu(γ-N₃)-L-Tyr-D-Trp-L-Lys-L-Thr-L-amino-5-pentanoic-acid-L-Thr-ol].

The peptide was prepared in a Teflon reactor with a porous polystyrene septum, using the Fmoc/tBu SPPS strategy on pre-swollen H-L-Thr(t-Bu)-ol-2-chlorotrityl resin.

The coupling steps were carried out adding 2 eq. of protected amino acids, activated with HATU in case of unnatural synthetic amino acid (ω-alkynyl and ω-azido) and HOBt/HBTU (Hydroxybenzotriazole/_{—}2(1H-Benzotriazole-1-yl)-1,1,3,3-tetramethyluronium tetrafluoroborate) in other cases, and 4 eq. NMM in DMF, then stirred for 45 minutes and followed by Kaiser test monitoring. Fmoc-L-Lys-(Boc)-OH was coupled in position 5.

At the end of the synthesis the resin was treated with Trifluoroacetic acid/H₂O/1,2-Ethandithiol/Phenol (94:2:2:2) for 3 hours. This mixture allowed to cleave the peptide from the resin and simultaneously deprotecting all acid sensitive amino acid side-chains protecting groups. The solution was concentrated, the peptide was precipitated with Et₂O, filtered, dissolved in water and lyophilized. Analysis by RP-HPLC using Kinetex™ 2.6 μm C18 100 Å LC Column 150×3 mm, method 10-60% of B in A for 5 min (A=0.1% TFA in H₂O, B=0.1% TFA in CH₃CN) of the crude peptide has shown the presence of the linear 95% pure linear conjugated peptide at Rt=4.09 min [M+H]⁺1452.51.

### Example 5

Synthesis of 1,4-disubstituted-[1,2,3]triazolyl bridge containing conjugate octapeptide formula II were A is DOTA; m=n=2; Xaa=—CH—CH₂—C₆H₅—OH; Yaa=—CH—CH(OH)—CH₃; T=1,4-disubstituted-[1,2,3]triazol (formula I).

### On Resin Strategy

Parent linear heptapeptide was synthesized as described above accordingly to the Fmoc/tBu SPPS strategy.

The on-resin linear heptapeptide was subjected to cyclization step. The cyclization of the peptide was carried out on the peptide linked to the resin by CuI-catalyzed azide-alkyne 1,3-dipolar Huisgen's cycloaddition to form regioselective 1,4-disubstituted-[1,2,3]triazolyl bridge.

The peptidyl resin (250 mg) was swollen for 2 hours in DMC/MeOH 1/1. CuI (0.5 eq.) and DIPEA (40 eq.) were added under nitrogen fluxing into the suspended resin.

The suspension was left at r.t. for 15 hours. Conversion of the linear precursor into the 1,4-disubstituted-[1,2,3]triazolyl-containing peptide was monitored by microscale cleavage on the No terminal amino acid Fmoc-deprotected peptidyl resin. The crude cyclo-heptapeptide was analyzed by RP-HPLC using Kinetex™ 2.6 μm C18 100 Å LC Column 150×3 mm, method 10-60% of B in A for 5 min (A=0.1% TFA in H₂O, B=0.1% TFA in CH₃CN) showing complete conversion of linear precursor into cyclic one (Rt of cyclo-heptapeptide=3.69 minutes).

All resin amount was then treated with 20% piperidine in DMF and the Fmoc group of the azido amino acid was removed. Subsequently 2 eq. of Fmoc-D-Phe-OH in DMF, 2 eq. HOBt/HBTU (Hydroxybenzotriazol/2-(1H-Benzotriazol-1-yl)-1,1,3,3-tetramethyluronium tetrafluoroborate) and 4 eq. NMM were added to the resin. Fmoc group of the Na terminal amino acid D-Phe was removed and 2 eq. of DOTA-tris-(^{t}Bu)-ester (CheMatech) in DMF, 2 eq. of HATU (O-(7-Azabenzotriazol-1-yl)-N,N,N′,N′-tetramethyluronium hexafluorophosphate) and 4 eq. of NMM were added to the resin. The 1,4-disubstituted-[1,2,3]triazolyl-containing conjugated-octapeptide was cleaved as described above with the simultaneous deprotection of ^{t}Bu groups of the DOTA moiety.

The crude peptide was dissolved in water and lyophilized, then purified by semi-preparative RP-HPLC 20-50% of B in A for 20 min (A=0.1% TFA in H₂O, B=0.1% TFA in CH₃CN). Analysis of purified peptide with Kinetex™ 2.6 μm C18 100 Å LC Column 150×3 mm method 20-60% of B in A for 5 min A=0.1% TFA in H₂O, B=0.1% TFA in CH₃CN) shows the peptide 1,4-disubstituted-[1,2,3]triazolyl-containing conjugated-octapeptide was >95% pure Rt=3.11 minutes, [M+H]⁺1452.51.

### Example 6

Synthesis of 1,4-disubstituted-[1,2,3]triazolyl bridge containing conjugate octapeptide formula II were A is DOTA; nn=n=2; Xaa=—CH—CH₂—C₆H₅—OH; Yaa=—CH—CH(OH)—CH₃; T=1,4-disubstituted-[1,2,3]triazolyl group.

### In Solution Strategy

Synthesis of parent linear octapeptide was carried out as described in Example 5 anchoring at position 5 Fmoc-L-Lys(Dde).

The Fmoc Nα protecting group of D-Phe-OH was deprotected in 20% piperidine in DMF and the linear peptide was cleaved from the resin as described above to obtain D-Phe-Abu(γ-N₃)-L-Tyr-D-Trp-L-Lys(Dde)-L-Thr-L-amino-5-pentanoic-acid-L-Thr(ol).

Heterodetic cyclooctapeptide was generated in solution by intramolecular Cu(I)-catalyzed azido-alkyne 1,3-dipolar Huisgen's cycloaddition, in tBuOH/H₂O as solvent mixture, in the presence of 5 eq. of ascorbic acid and 5 eq. of Cu₂SO₄ generating in situ Cu(I). The crude cyclooctapeptide was analyzed by RP-HPLC (Kinetex™ 2.6 μm C18 100 Å LC Column 150×3 mm, method 20-60% of B in A for 5 min A=0.1% TFA in H₂O, B=0.1% TFA in CH₃CN), showing complete conversion of the linear precursors (Rt shift from linear 4.0 minutes to heterodetic cyclootapeptide 3.5 minutes).

The DOTA chelating group was anchored using 4 eq. of DOTA, 5 eq. of NHS, 5 eq. of EDCI and 8 eq. of DIPEA in a mixture of water/DMF. The solvent was evaporated and methanol was added to the crude. The suspension was centrifuged and the solid discarded. The peptide dissolved in methanol was precipitated using Et₂O. Finally the Dde-protecting group on Lys was removed dissolving the peptide into 2% hydrazine hydrate in DMF. The peptide was then precipitated using Et₂O. The crude heterodetic conjugated-cyclooctapeptide was dissolved in water and lyophilized, then purified by semi-preparative RP-HPLC 20-50% of B in A for 20 min (A=0.1% TFA in H₂O, B=0.1% TFA in CH₃CN). Analysis of purified peptide with Kinetex™ 2.6 μm C18 100 Å LC Column 150×3 mm method 20-60% of B in A for 5 min A=0.1% TFA in H₂O, B=0.1% TFA in CH₃CN) shows the peptide 1,4-disubstituted-[1,2,3]triazolyl-containing conjugated-octapeptide was >95% pure Rt=3.11 minutes, [M+H]⁺ 1452.51.

### Example 7

¹¹¹In-Radiolabeling of linear conjugated octapeptide of Formula (I) where A is DOTA; m=n=2; Xaa=—CH—CH₂—C₆H₅—OH; Yaa=—CH—CH(OH)—CH₃; R1=—N═N⁺═N⁻; R2=alkynyl

20 MBq of ¹¹¹InCl₃ ([¹¹¹In]indium chloride (¹¹¹InCl₃, 370 MBq.mL⁻¹ in 0.05 N HCl) purchased from Perkin Elmer) were added to 12 μg of the DOTA-peptide in 0.1 M ammonium acetate buffer, pH 5.7, to reach a buffer/HCl (from ¹¹¹InCl₃ solution) ratio of 1.5:1 resulting in a pH 5 solution (Specific activity 2.36 MBq/nmol). The reaction was achieved in 1 hour at +75° C. and gave a radiolabeling yield of 96% before purification, determined by radio-HPLC. HPLC analyses were performed on a Kinetex™ column 2.6 μm C18 100 Å LC Column 2.10×50 mm, coupled with a Flow-Count radio-HPLC Detection System (Bioscan10). Crude product was then purified through a Sep Pack® C18 cartridge preconditioned as described in the literature. Free Indium was removed with water and the ¹¹¹In-DOTA-Peptide was eluted with a mixture of EtOH/PBS (7:3). The pure fractions were analyzed by radio-HPLC. ¹¹¹In-DOTA-peptide was obtained with a radiochemical purity >98%.

### Example 8

¹¹¹In-Radiolabeling of linear conjugated octapeptide of Formula (I) where A is DOTA; m=n=2; Xaa=—CH—CH₂—C₆H₅—OH; Yaa=—CH—CH(OH)—CH₃; R1=alkynyl, R2=—N═N⁺═N⁻

20 MBq of ¹¹¹InCl₃ ([¹¹¹In]indium chloride (¹¹¹InCl₃, 370 MBq.mL⁻¹ in 0.05 N HCl) purchased from Perkin Elmer) were added to 7.35 μg of the DOTA-peptide in 0.1 M ammonium acetate buffer, pH 5.7, to reach a buffer/HCl (from ¹¹¹InCl₃ solution) ratio of 1.5:1 resulting in a pH 5 solution (Specific activity 5 MBq/nmol). The reaction was achieved in 1 hour at +75° C. and gave a radiolabeling yield of 98% before purification, determined by radio-HPLC. HPLC analyses were performed on a Kinetex™ column 2.6 μm C18 100 Å LC Column 2.10×50 mm, coupled with a Flow-Count radio-HPLC Detection System (Bioscan®). Crude product was then purified through a Sep Pack® C18 cartridge preconditioned as described in the literature. Free Indium was removed with water and the ¹¹¹In-DOTA-Peptide was eluted with a mixture of EtOH/PBS (7:3). The pure fractions were analyzed by radio-HPLC. ¹¹¹In-DOTA-peptide was obtained with a radiochemical purity >99%.

1-19. (canceled)

20. New conjugated somatostatin analogs of formula [I], 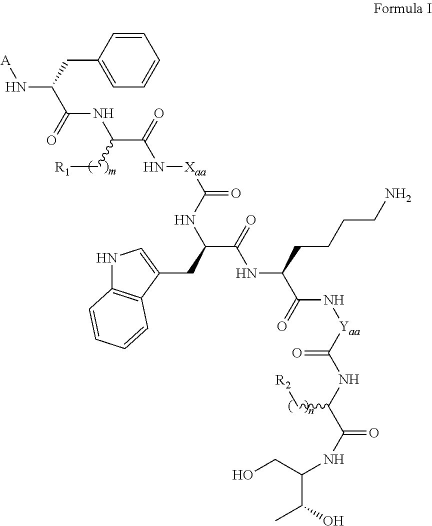
wherein A- is H or TAG-B—,
wherein —B— is 0 or a spacer of formula C-D-E,
—C— and E are the same or different and are:
a chain —(CH₂)ᵢ—W where i ranges from 0 to 20 and can be preferably 0 and 1 and W is 0, a chain containing 1 to 6 (C═O) or (C═S) group(s) and/or one or more heteroatoms (N, O, S, P), and/or one or 1 to 3 aromatic moiety(ies) containing zero, one or more heteroatoms (N, O, S, P), ortho and/or meta and/or para substituted or a chain containing one to 12 amino acid moieties choosing among the 22 natural α-amino adds, β or γ amino acids residues, preferably lysine or threonine.
a (PE_{G})ⱼ-W chain where j=2, 4, 100, 300, 1000 and W is as described above
D being a group resulting from the coupling of two functional groups F and G present on the TAG and on the peptide respectively and defined as follows:
D=thiourea generated from F=—NH₂ and G=—N═C═S (and vice versa), D=amide generated from F=NH₂ and G=COX (X=activated ester, Cl, anhydride, . . . ) (and viceversa), D=thioether generated from F=maleimide- and G=—SH (and vice versa); D=1,4-disubstituted [1,2,3]-triazole generated from F=—N═N⁺═N⁻ and G=-alkyne (and viceversa), D=imine generated from F=aldehyde and G=NH₂ (and vice versa), D=coupling function generated from F=norbornene or trans-cyclooctene and G=1,2,4,5 tetrazine (and viceversa),
wherein TAG is
a chelating agent CA
wherein CA-B is one of the formulae 1 to 8,
R and R′ are groups capable of chelating a metal of interest, for either imaging purposes (Gd (MRI), ¹¹¹In or ⁶⁷Ga (SPECT), ⁶⁴Cu or ⁶⁸Ga (PET), . . . ) or therapy (⁹⁰Y, ¹⁷⁷Lu, ⁶⁷Cu, . . . ), preferentially COO⁻, COOH, P(O)(OH)Me or CONH₂ 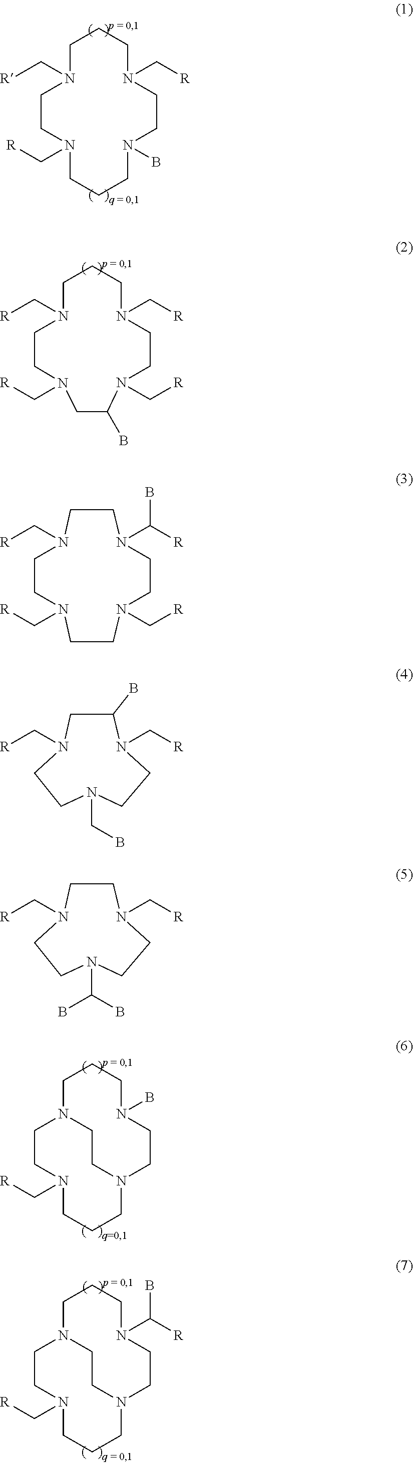 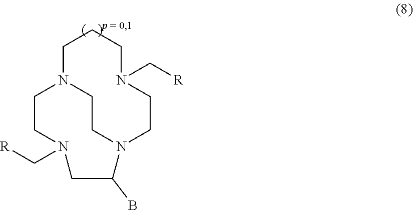
a fluorescent dye FD
wherein FD-B is an organic fluorophore-B such as a Bodipy-B derivative, a Rhodamine-8 derivative, a Fluorescein-B derivative, a Cyanine-B derivative, a Porphyrin-B derivative or a fluorescent complex of formulae (9) to (12) wherein J is a chromophore group and Ln is a Lanthanide chosen from Lanthanides giving high fluorescent Lanthanide complexes. 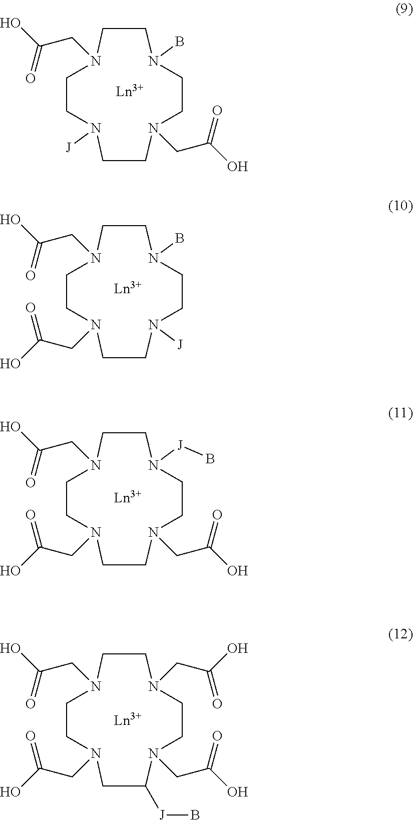
a bimodal agent BA comprising both a chelating agent CA and a fluorophore FD coupled together particularly using an amino acid linker, for example BA-B being one of the formula 13 or 14 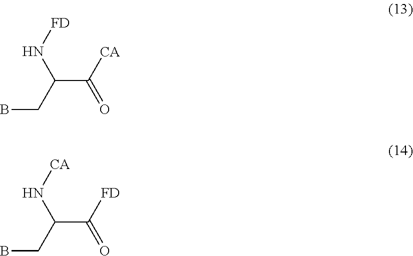
FD and B are the same as described previously,
a cytotoxic molecule CT
wherein CT-B is a potent cytotoxic derivative of 2-pyrrolino-DOX (formula 15) or doxorubicin (DOX) of formula (16) capable to inhibit the growth of various tumors, 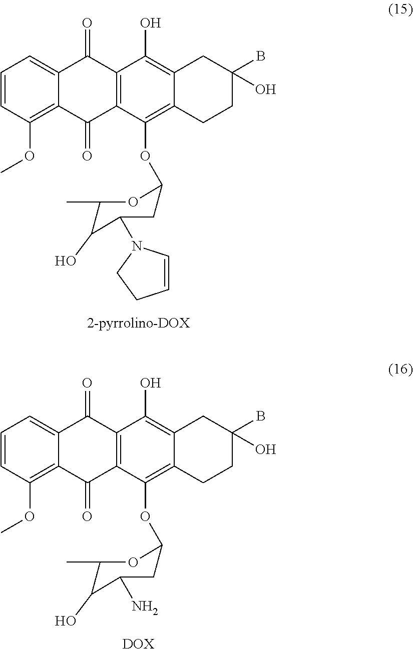
wherein Xaa is all at least one of the 22 L or D amino adds or phenylalanin either unsubstituted or ortho- or para-substituted with the OR″ group where, R″ is linear or branched C1-C6 alkyl substituted; otherwise Xaa is 1-NaI (NaI=naphthylalanine) or 2-NaI as such substituted on the 1 or 2 ring by OR″ group where R″ is H or linear or branched C1-C6 alkyl substituted or a C6H₅—CH₂—,
wherein Yaa is all 22 L, D amino acids or phenylalanine either unsubstituted or ortho- or para-substituted with the OR″ group where, R″ is linear or branched C1-C6 alkyl substituted or a threonine substituted with OR′″ where R′″ is H or a linear branched alkyl residue or a C₆H₅—CH₂— residue either unsubstituted or ortho- or para-substituted with the OR″ group, R″ being as above defined or a substituted C₆H₅—CH₂— or 1- or 2-naphthylmethyl-; otherwise Yaa 1-NaI or 2-NaI as such unsubstituted or substituted on the 1 or 2 ring by —OCH₃ or C₆H₅—CH₂—O—;
wherein R1 is C₁-C₄ alkynyl radical when R2 is C₁-C₄ azido radical and viceversa.
m and n are the same or different and are 1 to 5.

21. Conjugated somatostatin analogs of claim 20 wherein R1 and R2 form an intramolecular 1,4 disubstituted [1,2,3]triazolyl bridge T, the resulting compounds having formula (II) 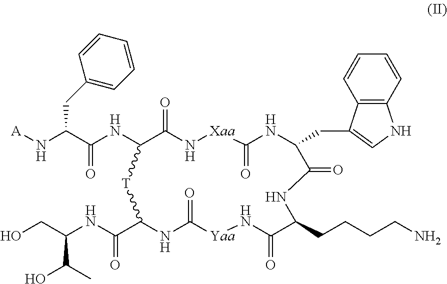
Wherein A, Xaa, Yaa are the same as described above. The orientation of the 1,4 disubstituted [1,2,3]triazolyl moiety depends on the position of ω-azido or ω-alkynyl aminoacids in the peptide chain, respectively i+5 and viceversa.

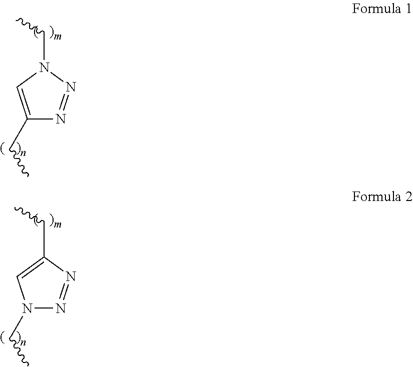

22. Conjugated somatostatin analogs of claim 20, wherein C is selected from the group comprising 0, CH₂—, CH₂CH₂—, CH₂Ar—, CH₂CH₂Ar—, CH₂CONHCH₂CH₂—, CH₂CONHCH₂CH₂NHCH₂—, CH₂NHCOCH₂Ar—, CH₂NHCOCHArNHCOCH₂—, CH₂NHCH₂ArOCH₂—.

23. Conjugated somatostatin analogs of claim 20, wherein E is selected from a group containing one amino acid moiety chosen among the 22 natural α-amino acids, β or γ amino adds residues or a group containing an unnatural amino acid modified on α amino or carboxyl group and/or in side chain position.

24. Conjugated somatostatin analogs of claim 20, wherein A represents H.

25. Conjugated somatostatin analogs of claim 20, wherein A represents TAG-B.

26. Conjugated somatostatin analogs of claim 20, wherein TAG-B is CA —B chelated with ¹¹¹In, ^{68/69}Ga or ⁶⁴Cu or FD-B or BA-B for imaging.

27. Conjugated somatostatin analogs of claim 20, wherein TAG-B is CA-B and chelated with for example ⁹⁰Y, ¹⁷⁷Lu or ⁶⁷Cu for therapy.

28. Conjugated somatostatin analogs of claim 20, wherein TAG is a cytotoxic molecule CT.

29. A method for preparing the conjugated somatostatin analogs of formula (I) according to claim 20 by Solid Phase Peptide Synthesis (SPPS), comprising
anchoring a peptidic sequence to a derivatized chlorotrityl resin
recovering the crude peptide by treating the resin for example with trifluoroacetic acid
separating by precipitation for example using ethyl ether
carrying out successive lyophilization
purifying the peptide using Chromatographic technique for example RP-HPLC.

30. The method of claim 29 further comprising, for preparing the derivatives of formula (II),
a) Synthesizing a linear heptapeptide following the Fmoc/tBu SPPS strategy with appropriate side chain protected aminoacids.
b) Cyclising the peptides linked to the resin by means of Cu(I)-catalyzed azide-alkyne 1,3-dipolar Hulsgen's cycloaddition to form regioselective 1,4-disubstituted-[1,2,3]triazolyl bridge
and either
c) adding the N-terminus aminoacid and the group A defined as described above in solid phase or alternatively following the solution strategy describe in step c′ to e′.
d) cleaving the conjugated 1,4-disubstituted-[1,2,3]triazolyl bridge containing peptides from the resin with an appropriate acid
e) purifying by semipreparative RP-HPLC the crude 1,4-disubstituted-[1,2,3]triazolyl containing cyclic conjugated peptides
or
c′) adding the N-terminus amino acids and/or the group E-G, defined as described above; cleaving the peptide from the resin with an appropriate acid.
d′) coupling the group TAG in solution to the 1,4-disubstituted-[1,2,3]triazolyl bridge containing peptides.
e′) purifying by semipreparative RP-HPLC the crude 1,4-disubstituted-[1,2,3]triazolyl bridge containing peptides conjugated.

31. The method of claim 20, further comprising, when A represents TAG-B (or TAG-C-D-E), conjugating the resulting peptide to a tag derivative of formula (III)
TAG-C—F (III)
wherein C and F are the same as described above.

32. Radiotracers for imaging tumoral cells, comprising at least one conjugated somatostatin analogs according to claim 20.

33. Compounds according to claim 20 used for optical imaging.

34. Radiotracers according to claim 20 used for SPECT/PET imaging or for nuclear/optical imaging.

35. A method of radioisotope imaging comprising the use of at least one compound according to formula (I) or (II) wherein A represents TAG-B.

36. Pharmaceutical compositions comprising a therapeutically efficient amount of at least one conjugated somatostatin analog of formula (I) in combination with a pharmaceutically acceptable carrier.

37. The pharmaceutical compositions of claim 36 wherein said conjugated somatostatin analog(s) is (are) used for the treatment of cancer.

38. The method of claim 37 wherein said analogs are used for the treatment of lymphoma, pancreatic, lung, prostate or breast cancer, or adenoma.
